(19) United States
(12) Plant Patent Application Publication
(10) Pub. No.: US 2009/0144870 P1
(43) Pub. Date: Jun. 4, 2009

(54) Beallara Tropic Tom 'Purple Paisley'

(50) ;

(76) Inventor: James McCully, Hilo, HI (US)

Correspondence Address: JAMES WILLIAM MCCULLY, 2146 KAUMANA DRIVE, HILO, HI, 96720, US

(21) Appl. No.: 11/999,336

(22) Filed: Dec. 3, 2007

(51) Int. Cl. A01H 5/00

(52) U.S. Cl. PLT/311

(57) ABSTRACT

A new variety of orchid plant of the Oncidiinae Intergenerics, Beallara, named Beallara Tropic Tom ‘Purple Paisley’, distinguished particularly by a compact size, double spiking psuedobulb, large star-shaped, purple shaded flowers that are arranged pleasingly on a raceme and growing quickly to sexual maturity.

### CROSS-REFERENCE TO RELATED APPLICATIONS

Not Applicable

### STATEMENT REGARDING FEDERALLY SPONSORED RESEARCH OR DEVELOPMENT

Not Applicable

### LATIN NAME OF THE GENUS AND GREX OF THE PLANT CLAIMED

The Latin name of the genus and grex of the plant claimed is Beallara Tropic Tom.

### VARIETY DENOMINATION

The present invention comprises a new and distinct cultivar of Beallara orchid, and hereinafter referred to by the cultivar name Beallara Tropic Tom ‘Purple Paisley’.

### BACKGROUND OF THE INVENTION

Beallara is an artificial hybrid of orchids between four genera in the subtribe Oncidiinae. Beallara is made from the genera Odontoglossum, consisting of about 300 species, Brassia, consisting of a little over 30 species, Miltonia, consisting of about 11 species of the ‘Brazilian Miltonias’ (intermediate to warm growing, distinguished from the cool growing ‘Miltoniopsis’) and Cochlioda, consisting of 6 species. The first hybrid of Beallara was created in 1970 by Beall and registered by Moir. Due to the varying percentages of the parent genera that make up Beallara and the gene expression in their progeny, Beallara varies in morphology and size. All exhibit sympodial growth habit.

Beallara breeding is typically done from sexual methods. Asexual propagation of Beallara is often done in aseptic tissue culture from apical or axillary shoots.

The new cultivar was discovered by the Inventor within the progeny of a cross made by the Inventor on Jan. 16, 2000. The Inventor selected Beallara Tropic Tom ‘Purple Paisley’ as a single plant from a population of over 50 plants grown by James McCully Orchid Culture Inc. dba Mauna Kea Orchids in Hilo, Hawaii, a company owned by the Inventor. Beallara Tropic Tom ‘Purple Paisley’ was submitted by the Inventor to a commercial tissue culture laboratory, Bangkok Flower Centre, Bangkok, Thailand on Jun. 28, 2004, for propagation through aseptic tissue culture technique. A quantity was produced for evaluation and has demonstrated that the unique combination of characteristics as herein disclosed for the new cultivar are firmly fixed and are retained through successive generations of asexual reproduction.

Other seedlings from the same cross that was used to produce Beallara Tropic Tom ‘Purple Paisley’ have been commercially available from others. The characteristics of Beallara Tropic Tom ‘Purple Paisley’ are clearly distinguishable from the characteristics of those other plants. Those other plants were sold by James McCully Orchid Culture without specific epithet to individuals and other nurseries. To the Inventor's knowledge, the only other named clones of the cross are Beallara Tropic Tom ‘Pale Face’, Beallara Tropic Tom ‘Purple Valentine’ and Beallara Tropic Tom ‘Tom Terrific’. The inventor has reason to believe that Beallara Tropic Tom ‘Purple Paisley’ is superior to these other clones in size of flowers, color of flowers and spiking habits. Plants of the grex Beallara Tropic Tom are sold as plants of the grex without specific epithet.

### BRIEF SUMMARY OF THE INVENTION

The following traits have been repeatedly observed and are determined to be basic characteristics of the new cultivar which in combination distinguish this Beallara orchid as a new and distinct cultivar:

Flowers of the new cultivar are cream in color with irregular maroon blotches and purple picotee on the sepals and petals. The labellum is bright purple with a yellow callus and becomes overlaid with maroon blotches towards the apices. The anther cap and column are cream in color, the column densely striated purple. The natural horizontal spread is 10.5 centimeters (cm); natural vertical spread is 11.2 cm; sepal width 1.6 cm; sepal length 5.5 cm; petal width 1.9 cm; petal length 5.5 cm; labellum width 4.4 cm; labellum length 5.5 cm. The plant produces one or two inflorescences on the first bloom with four to seven flowers on each.

Inflorescence is a basal raceme, upright or arching, and approximately 30-50 cm tall. The plant is relatively compact and the size of the inflorescence is appropriate to the overall size of the plant and typical pot size for this cultivar.

There are generally three leaves on the mature psuedobulb, one from the apex and two from the base of the psuedobulb. The apical leaves measure around 42.7 cm and are around 5.3 cm wide. The leaves from the base of the pseudobulb measure up to 34.5 cm and are 4.2 cm wide. Axillary leaves, or bracts, are usually present below the leaves at the base of the psuedobulb. When present, there is usually one on either side of the pseudobulb, 7 to 15 cm long, appearing as a sheath. One side is almost always twice as large as the other side. The shape of the leaf is lanceolate acuminate and they are conduplicate at the base, especially the apical leaves. The texture and substance is undulate, waxy and somewhat coriaceous. The plants of Beallara Tropic Tom ‘Purple Paisley’ grow to maturity and flower in approximately 24 months.

Plants of the new cultivar have not been observed under all possible environmental conditions. The phenotype may vary significantly with variations in the environment such as temperature, light intensity, and day length, without however any change in genotype.

Plants of the new cultivar differ primarily from the plants of the parent cultivars in flower color, panicle size, rate of growth and speed to maturation. Perhaps the closest commercial comparison to the new cultivar can be made to seedling-derived Beallara orchids which are genetically heterogeneous, and typically lack uniformity in growth, vigor, plant habit, and flower quality. Since this reference point has inconsistent characteristics, a direct comparison for Beallara Tropic Tom ‘Purple Paisley’ is not available. The new cultivar is a single genotype asexually propagated via tissue culture; thus, its combined horticultural characteristics listed above are uniform and predictable.

### BRIEF DESCRIPTION OF THE DRAWINGS

The drawings show the colors of the new variety as nearly true as possible with an illustration of this type. In the drawings:

FIG. 1 is a view of the entire plant to reveal the growth habit and general characteristics with its flowers, which is typical of this new variety.

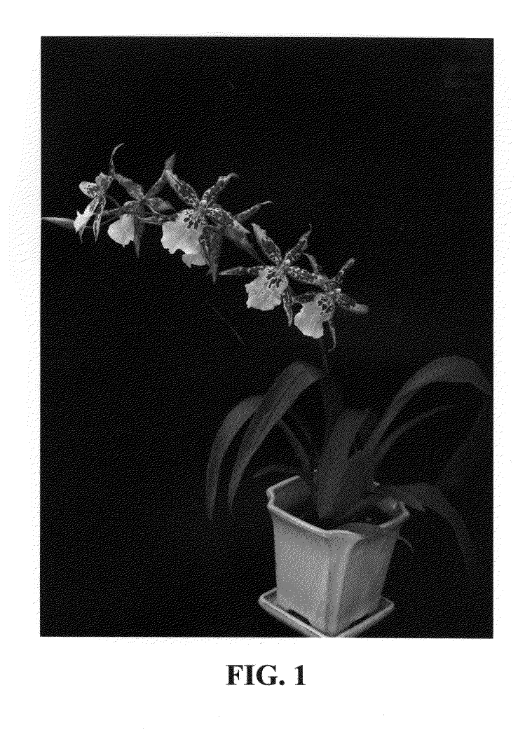

FIG. 2 is a close up view of the flower to show its shape.

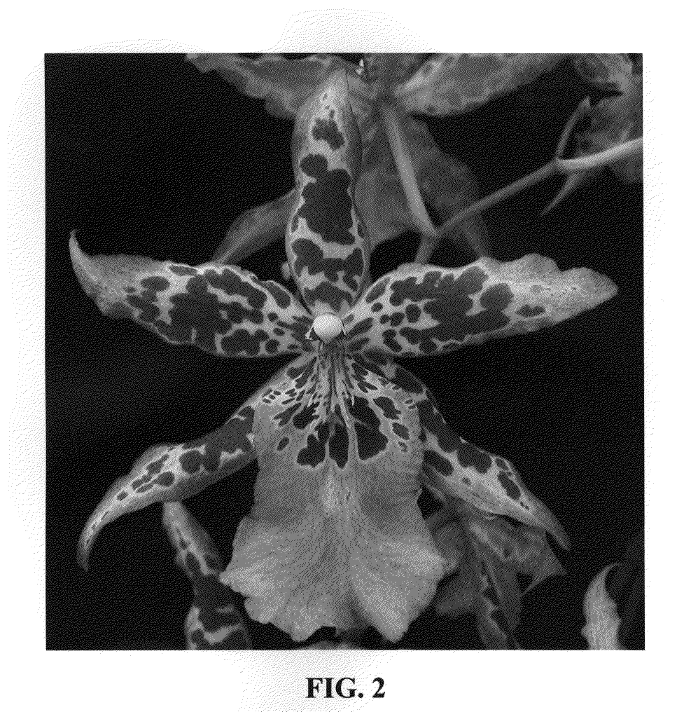

### DETAILED BOTANICAL DESCRIPTION

All color references are measured against the Pantone® Color System. Colors and numerical measurements are approximate as plant growth and development depends on environmental conditions and cultural practices such as light level, temperature, water quality, fertilization formula and rate, among others, without, however, any variance in genotype.

Plants used for this description are 2 years in vivo and grown in 4 inch, square, black plastic pots, grown in a poly-carbonate covered greenhouse in Hilo, Hawaii, where day temperatures range from 72 to 88 degrees and night temperatures range from 56 to 72 degrees Fahrenheit. Light levels are between 1500 to 2500 foot candles. This information reflects the annual variations for the area.
Botanical classification: Beallara Tropic Tom ‘Purple Paisley’
Parentage: Seedling selected from the cross of the following:
   Seed parent.—Beallara Tropic Splendor.
   Time to initiate and elongate roots in-vivo.—about 45 days.
   Time to produce fully rooted young plant.—about 285 days.
Root description: Velamen covered roots, approximately 2 cm in diameter, white in color with yellow to green growing tip. The exact shades of white and yellow/green vary greatly with minimal changes in environmental conditions.
Plant description: Under appropriate growing conditions, plants of the new cultivar attain a mature size of about 51 cm in height (top of leaf plane) and about 5.6 cm in width.
Foliage description:
   Arrangement.—Sympodial growth habit.
   Quantity of leaves per growth.—Each sympodial growth has shown three leaves, one on the apex of the pseudobulb and two from the axis at the base of the pseudobulb.
   Leaf length.—Leafs measure up to 42.7 cm in length. The axillary leaves at the base of the psuedobulb may be as short as 7 cm to appear to be a sheath and as long as 15 cm.
   Leaf width.—The width of the largest leaf measures up to 5.7 cm.
   Pollen parent.—Odontioda Marside.
Propagation:
   Type.—Asexual propagation by aseptic tissue culture through axillary shoot initiation.
   Leaf shape.—The shape is lanceolate acuminate, texture and substance is undulate, waxy and coriaceous.
   Pseudobulb shape.—The pseudobulb shape is a laterally compressed ovoid.
   Pseudobulb length.—The pseudobulb is approximately 8 to 12 cm long.
   Psuedobulb width.—The pseudobulb is approximately 4 cm wide and 1.5 cm thick.
   Foliage color.—Under the described growing conditions, the foliage color on both sides of the leaves is similar to Pantone® 378UP (green) of the Pantone® Color System. There is no vein color distinction. The pseudobulb is also Pantone® 378UP (green).
Flower/inflorescence description:
   Description.—Upright or arching raceme, approximately 49.3 cm on a first bloom plant and has 1 or 2 racemes from a single psuedobulb with 4 to 7 flowers on each raceme. The flowers begin approximately 20 cm up the raceme and are spaced around 4.5 cm apart at the lower flowers. Towards the top of the raceme, the flowers are more densely spaced, from 2.5-3.5 cm apart. The size of the inflorescence is appropriate to the overall size of the plant and typical pot size for this cultivar.
   Dimensions.—Flower dimensions are: natural horizontal spread is 10.5 cm; natural vertical spread is 11.2 cm; sepal width is 1.6 cm; sepal length is 5.5 cm; petal width is 1.9 cm; petal length is 5.5 cm; labellum width is 4.4 cm; labellum length is 5.5 cm.
   Coloration.—The dominant flower color that appears on the edges of the sepals and petals as well as the labellum ranges in shades of purple from Pantone® 257U to Pantone® 258U. The interior color of the tepals are cream Pantone® 7499PC and they are heavily blotched maroon, Pantone® 229UP. The labellum is purple, Pantone® 257U to Pantone® 258U, and is apically blotched maroon, Pantone® 229UP. The crest of the labellum is bright yellow, Pantone® 128C with a few small and irregular lines of maroon, Pantone® 229UP. The anther cap is cream, Pantone® 7499PC. The base color of the column is cream, Pantone® 7499PC and is densely striated purple, Pantone® 7499U on top and Pantone® 7434UP on the sides. The pollinia are completely concealed by the anther cap, and if it is removed the pollinia may be extracted. The pollinia are yellow, Pantone® 116U, but can range in shades of yellow as the plant ages.
Quantity of flowers and time to flower:
   Flower longevity.—The flowers can last up to 8 weeks on the plant with good environmental conditions. A first bloom plant will produce 5-12 flowers depending on environmental conditions.
   Natural flowering season.—The natural flowering season is early fall.
Fragrance: No fragrance has been detected by the Inventor.
Reproductive organs: The stamens, style and stigmas are fused into a single short structure called the column, possessing one terminal anther with pollen grains united to a pollinia, which are covered by an anther cap. The stigma is located under the column behind the pollinia. The ovary is inferior, with three carpels being present.
   Column.—The column is erect with wings on either side of the stigma, 8 mm wide by 1.2 cm long.
   Pollinia.—Two oval masses of pollen are present, about 1 mm in diameter.
   Stigma.—The stigma is concave, round, with a high gloss, and sticky.
   Ovary.—The ovary is about 3.5 cm long by 5 mm in diameter.
   Seed.—Seed production has not been observed.
Disease resistance: Resistance or susceptibility to known pathogens of Beallara has not been observed on plants grown under commercial production conditions.
General observations: Plants of Beallara Tropic Tom ‘purple paisley’ produce a pleasing arrangement of large star-shaped flowers of purple shades on a raceme that is desirable in size for the size of the plant. The flowers are long lasting and nicely shaped. The plant grows quickly to sexual maturity.

What is claimed is:

1. A new and distinct variety of orchid plant named Beallara Tropic Tom ‘Purple Paisley, substantially as illustrated and described herein.
